(19) United States
(12) Patent Application Publication
(10) Pub. No.: US 2026/0250731 A1
(43) Pub. Date: Aug. 27, 2026

(54) METHOD OF IMPROVED CELLULOSE HYDROLYSIS

(71) Applicant: Worcester Polytechnic Institute, Worcester, MA (US)

(72) Inventors: Michael T. Timko, Arlington, MA (US); Maksim Tyufekchiev, Worcester, MA (US); Ali A. Salifu, Worcester, MA (US); Winston Soboyejo, Northborough, MA (US); Nneka B. Ekwe, Worcester, MA (US); Ziyang Zhang, Worcester, MA (US)

(21) Appl. No.: 18/997,625

(22) PCT Filed: Mar. 27, 2023
(86) PCT No.: PCT/US2023/016410

§ 371 Date: Jan. 22, 2025

Related U.S. Application Data
(60) Provisional application No. 63/324,370, filed on Mar. 28, 2022.

(51) Int. Cl. C12P 19/02; C12P 19/14; C13K 1/02

(52) U.S. Cl. CPC C12P 19/02; C12P 19/14; C13K 1/02; C12P 2201/00

(57) ABSTRACT

An example method of hydrolyzing cellulose includes: providing cellulose to be hydrolyzed; applying a stabilizing salt to the cellulose to form a reaction mixture, the stabilizing salt configured to suppress recrystallization of amorphous cellulose in the reaction mixture; and hydrolyzing the amorphous cellulose in the presence of the stabilizing salt to suppress recrystallization of the amorphous cellulose in the reaction mixture during hydrolysis.

### FIELD

The specification relates generally to cellulose hydrolysis, and more particularly to an improved method of cellulose hydrolysis using a stabilizing salt.

### BACKGROUND

Biofuels are commonly sourced from edible feedstock and would be more sustainable if produced from non-edible feeds. However, non-edible feeds are rich in lignocellulose, which must be broken down into fermentable sugars to yield useful chemical liquid and fuel products. Current methods of hydrolyzing cellulose to obtain fermentable sugars are expensive, inefficient, and energy intensive.

### SUMMARY

According to an aspect of the present specification an example method includes: providing cellulose to be hydrolyzed; applying a stabilizing salt to the cellulose to form a reaction mixture, the stabilizing salt configured to suppress recrystallization of amorphous cellulose in the reaction mixture; and hydrolyzing the amorphous cellulose in the presence of the stabilizing salt to suppress recrystallization of the amorphous cellulose in the reaction mixture during hydrolysis.

### BRIEF DESCRIPTION OF DRAWINGS

Implementations are described with reference to the following figures, in which:

FIG. 1A depicts a schematic diagram of cellulose having a crystalline structure.

FIG. 1B depicts a schematic diagram of cellulose having a partially amorphous structure.

FIG. 2 depicts a flowchart of an example method of hydrolyzing cellulose with a stabilizing salt.

FIG. 3 is a graph showing the x-ray diffractometer (XRD) results for fixed anion salt selections.

FIG. 4 is a graph showing the XRD results for fixed cation salt selections.

FIG. 5 is a graph showing the glucose yields for fixed anion salt selections.

FIG. 6 is a graph showing the glucose yields for fixed cation salt selections.

FIG. 7 is another graph showing the XRD results for fixed cation salt selections.

FIG. 8 is a graph showing the results of a molecular dynamic simulation.

FIG. 9 is a graph showing the radical distribution of —OH in cellulose.

### DETAILED DESCRIPTION

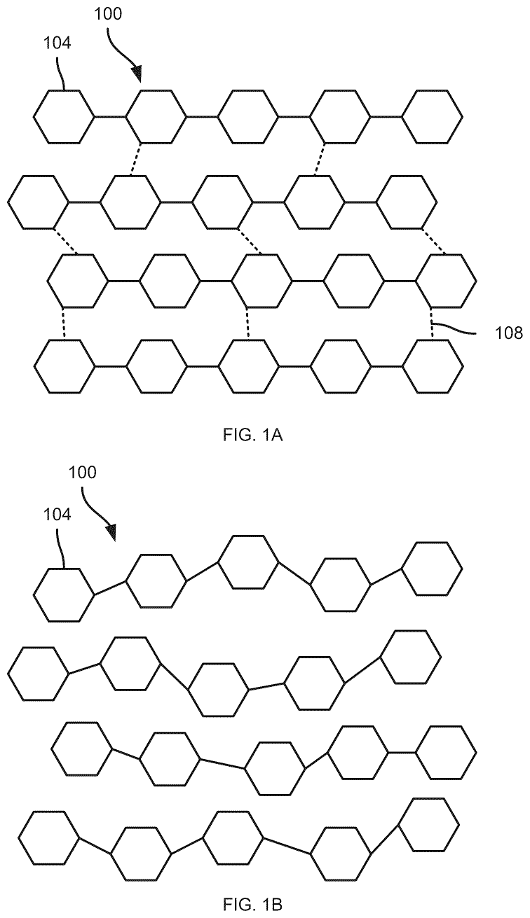

One of the challenges with hydrolyzing cellulose is its crystalline structure. Extensive hydrogen bonding yields a structural organization that prevents access by solvents and catalysts. For example, FIG. 1A depicts a schematic diagram of cellulose 100 having a crystalline structure. As can be seen, individual glucose units 104 form are generally inaccessible due to the hydrogen bonds 108 formed between glucose units 104 on different chains.

Decrystallization of the cellulose to be more amorphous increases accessibility by catalysts and reagents to the individual glucose units. For example, FIG. 1B depicts a schematic diagram of the cellulose 100 being in at least a partially amorphous state. That is, the cellulose 100, upon decrystallization, may separate into amorphous chains with substantially no hydrogen bonds therebetween, allowing catalysts or other reagents to access the individual glucose units. Thus, once the cellulose 100 becomes amorphous and disordered, reactivity with catalysts and other reagents increases.

Accordingly, crystalline cellulose may be treated by various means to decrystallize its structure and increase accessibility for hydrolyzation. For example, decrystallization treatments may include mechanical treatments, such as ball milling or other types of mechanical grinding, chemical treatments, such as dilute acid pretreatments, ionic liquid pretreatments, and the like.

In dilute acid pretreatment, biomass containing cellulose is exposed to a low concentration of acid at elevated temperatures of 140° C. to 210° C. for a few minutes or hours. This method is energy intensive, time consuming, and the conditions are harsh. In ionic liquid pretreatment, and other similar treatments, cellulose is treated with an ionic liquid to attempt to dissolve the cellulose into a completely amorphous form. However, such treatments may expose the cellulose to a variety of different chemicals which may not be compatible with enzymes or other catalysts or reagents subsequently used to hydrolyze the amorphous cellulose.

Unfortunately, decrystallization is temporary and cellulose molecules exposed to water will resume their crystalline structures. Furthermore, hydrolysis of the cellulose, for example via enzymatic or acidic treatments, can often precipitate recrystallization of the cellulose. That is, converting the cellulose into fermentable sugars encourages cellulose to reform the hydrogen bonds between glucose units. While some of the pretreatments outlined above may provide methods of decrystallizing the cellulose, many do not maintain cellulose in its decrystalline form during hydrolysis, resulting in lower glucose yields from biomass.

Accordingly, the present disclosure provides the use of a stabilizing salt applied to amorphous cellulose to suppress recrystallization of the amorphous cellulose. In particular, hydrolysis of the amorphous cellulose occurs in the presence of the stabilizing salt to suppress recrystallization of the cellulose during hydrolysis.

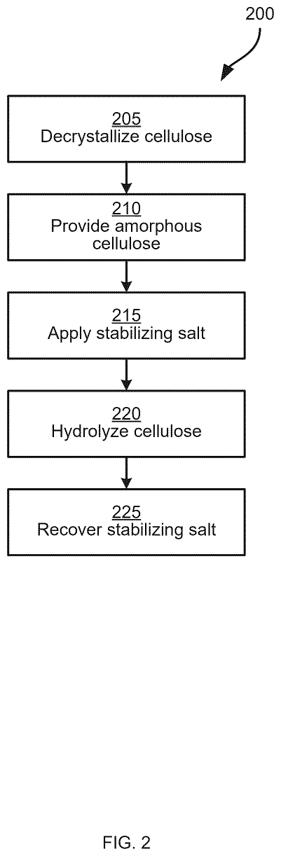

FIG. 2 depicts a method 200 of hydrolysis of a target biomass. As described herein, the method 200 is applied to cellulose as the target biomass, however in other examples, other similar polysaccharides or long-chain biopolymers having glycosidic bonds, such as, but not limited to, hemicellulose, lignocellulose, chitin, combinations of the above, and the like, may also be hydrolyzed according to the method 200.

At block 205, the cellulose (or other suitable target biomass) to be hydrolyzed may undergo decrystallization. Decrystallization of the cellulose may be achieved through a number of methods, including mechanical grinding, thermal and/or pressure change approaches, dilute acid and/or ionic liquid pretreatments, or other methods that cause the intramolecular hydrogen bonds in cellulose to release. In some examples, a combination of decrystallization approaches may be used to achieve decrystallization.

Preferably, decrystallization may be performed via mechanical grinding, such as by ball milling. This may, for example, reduce the potential for adverse chemical reactions and additional byproducts from the subsequent hydrolysis caused by introducing different reagents in dilute acid, ionic liquid, or other chemical-based pretreatment mechanisms. The mechanical grinding may be wet or dry.

At block 210, the decrystallized or amorphous cellulose is provided for a subsequent hydrolysis reaction. In some examples, the cellulose may be provided with a solvent or other carrier agent.

At block 215, prior to the hydrolysis of the cellulose, a stabilizing salt is applied to the cellulose to form a reaction mixture. In particular, the stabilizing salt is configured to stabilize the cellulose in its amorphous form in the reaction mixture, and to substantially suppress recrystallization of the cellulose.

The stabilizing salt may be a chaotropic salt, which generally disrupts hydrogen bonding and weakens the hydrophobic effect. The chaotropic effect of the stabilizing salt may therefore disrupt hydrogen bonding between glucose units to suppress recrystallization and promote stabilization of the amorphous glucose chains and suppress recrystallization of the glucose.

In particular, the stabilizing salt is preferably a salt comprising ions which are part of the Hofmeister series. The stabilizing salt may be selected according to its order in the Hofmeister series. In particular, the Hofmeister series classifies ions according to their “salting out” or “salting in” capacities. Low-order Hofmeister salts increase the solubility of nonpolar molecules and decrease the order in water. That is, as applicable to cellulose, low-order Hofmeister salts increase the solubility of cellulose, including amorphous or decrystallized cellulose, which may stabilize the cellulose in its amorphous form and suppress recrystallization. Accordingly, in some examples, the stabilizing salt may include a low-order Hofmeister salt.

For example, the Hofmeister salt utilized as the stabilizing reagent may be chosen from guanidine salts, salts of alkali metals, alkaline earth metal salts, and combinations thereof. More particularly, the salt may be guanidine salts of organic or inorganic acids, such as guanidine hydrochloride (GdmCl), guanidine thiocyanate, guanidine carbonate and guanidine phosphate; aminoguanidine salts such as aminoguanidine hydrochloride and aminoguanidine bicarbonate; salts of alkali metals such as lithium, such as lithium halides and more particularly lithium bromide; alkaline earth metal salts such as magnesium salts, such as magnesium halides; and combinations thereof. In further examples, the salt may be a guanidine salts, such as guanidinium halides, guanidinium thiocyanates, guanidinium carbonates; lithium salts such as lithium halides and preferably lithium bromide; and mixtures thereof. In particular examples, the salt is guanidinium chloride.

The particular type of stabilizing salt may be selected according to the selected subsequent hydrolysis method to be used to hydrolyze the glucose, based on compatibility of the salt with the acid, enzyme and/or other catalyst to be used for hydrolysis. For example, a salt which is prone to a salt-acid interaction during acid hydrolysis may provide lower stabilization capabilities during the hydrolysis operation. In other examples, the stabilizing salt may be selected to function in a suitable pH range, temperature range and other hydrolysis conditions as the selected enzyme during enzyme hydrolysis. Further the stabilizing salt may be selected to limit protein denaturization of the enzyme.

In some examples, the stabilizing salt may form a part of an aqueous solution to be applied to the cellulose. Accordingly, the stabilizing salt may particularly be to suppress recrystallization of the cellulose in the presence of water and/or during the subsequent hydrolysis operation. In other examples, the stabilizing salt may promote enzyme binding to expose the cellulose during hydrolysis.

The concentration of the stabilizing salt in the aqueous solution may depend on the particular salt used and/or the subsequent hydrolysis method to be used to hydrolyze the glucose. That is, the concentration of the stabilizing salt may be selected according to the selected method of hydrolyzing the amorphous cellulose in the reaction mixture. In some examples, the amount of the stabilizing salt ranges from 0.05 M to 1 M. In reactions where the method of hydrolysis is acid hydrolysis, the concentration of the stabilizing salt may be between 0.1 M and 1 M, and preferably between 0.4 M and 0.6 M. In reactions where the method of hydrolysis is enzymatic hydrolysis, the concentration of the stabilizing salt may be between 0.05 M and 0.4 M and preferably 0.2 M. In particular, the concentration of the salt may be sufficiently low so as to limit protein denaturization.

In some examples, for example where the decrystallization includes wet grinding, application of the stabilizing salt at block 215 may be performed substantially simultaneously to blocks 205 and 210. That is, the stabilizing salt may be applied to the cellulose during the decrystallization operation to limit the amount of immediate recrystallization. In other examples, application of the stabilizing salt at block 215 may be performed substantially simultaneously to block 220, as described below.

At block 220, the amorphous cellulose in the reaction mixture created at block 215 is hydrolyzed in the presence of the stabilizing salt to suppress recrystallization during the hydrolysis. The cellulose may be hydrolyzed, for example by enzymatic hydrolysis, acid hydrolysis, chemical hydrolysis, thermal hydrolysis, thermochemical hydrolysis, the like, or combinations thereof. Hydrolyzing the cellulose yields fermentable sugars such as glucose, xylose, and the like, and combinations thereof. These fermentable sugars may be converted into biofuel through various methods generally known in the art.

The hydrolysis operation occurs in the presence of the stabilizing salt to suppress recrystallization of the amorphous cellulose in the reaction mixture. In particular, the stabilizing salt may increase the solubility of the amorphous cellulose in the reaction mixture sufficiently to suppress recrystallization during hydrolysis. That is, the stabilizing salt may disrupt the formation of hydrogen bonds between glucose units which may be precipitated by hydrolysis. In other examples, the stabilizing salt may otherwise affect the crystallinity of the cellulose at the solid-liquid interface of the cellulose in the reaction mixture to suppress recrystallization.

In some examples, the stabilizing salt may further influence the binding of the enzymes used in enzymatic hydrolysis, or other catalysts used in other catalyst-promoted hydrolysis, to the amorphous cellulose, thereby promoting completion of the hydrolysis operation and limiting recrystallization.

Optionally, at block 225, after hydrolyzing the cellulose and isolating the fermentable sugars from the reaction mixture, the remainder of the reaction mixture may be fermented to recover the stabilizing salt. For example, a fermenting agent may be applied to the reaction mixture to recover ethanol, water, and the stabilizing salt. The fermenting agent may be selected according to its compatibility with the stabilizing salt. For example, the fermenting agent may include various types of yeast and bacteria, including, but not limited to, Saccharomyces cerevisiae or E coli. In particular, some salts may affect the effectiveness of the fermentation by the fermenting agent. Accordingly, the fermenting agent is selected based on its effectiveness in the presence of the stabilizing salt to allow recovery of the stabilizing salt.

As described herein, an improved method of biomass hydrolysis for hydrolyzing biomass components, including cellulose, hemicellulose, lignocellulose, and the like, includes application of a stabilizing salt and hydrolyzing the target biomass in the presence of said stabilizing salt to suppress recrystallization of the target biomass during hydrolysis. The addition of the stabilizing salt maintains the target biomass in its amorphous form, which provides better access for solvents and catalysts or other reagents during the hydrolysis step. Thus, the stabilizing salt improves yields of fermentable sugars and decreases processing times from the hydrolysis process.

The following examples provide experimental results pertaining to the above-described method of hydrolyzing a target biomass, and in particular, cellulose.

### Materials

In the examples described below, the cellulose samples comprise microcrystalline cellulose (Avicel PH-101, 50 μm particle size, Fluka Analytical®).

### Ball-Milling Pretreatment

In the examples described below, 1 gram of Avicel microcrystalline cellulose is ball-milled in a stainless steel cylinder (18 mm diameter×55.5 mm length, 10 mL). with three stainless steel balls (2×9.5 mm diameter and 1×15.85 mm diameter) placed in the cylinder. The cylinder is clamped within the holder of a vibratory shaker Retsch MM2000 and the sample is shaken for predetermined period of time. After the treatment the solids are analyzed with X-ray diffraction to determine the crystallinity of the substrate.

### Oil-Bath Temperature Calibration

In the examples described below, an Optichem heat and stirrer plate was used. The setup for all of the experimental runs had three test tube clamps set equally distant from the center of the silicone oil bath and at the same heights. The target internal sample temperature was 150° C. The final setting temperature for the oil bath was 173° C. The calibration was performed by using a k-type thermocouple that had been modified onto a pressurized cap. 2 mL of 0.05M HCl was used in the vessel as a volume control. The modified thermocouple vessel was place at equal heights above the oil bath, such that the liquid was fully submerged below the oil. A time study was performed on the liquid as it heated to the set temperature. This was repeated for each location to be used in the oil bath to ensure proper temperature had been reached. As stated, 173° C. was the final temperature setting for the Optichem® heat plate for an internal temperature of 150° C. to be reached. The data from this calibration was used to include a 10-minute warmup time to ensure the temperature of the sample was at 150° C. for the entirety of the time desired.

### Acid Hydrolysis

In the examples described below, 0.25 g of milled (or untreated) cellulose are mixed with 5 mL of 0.1 HCl and a magnetic stir bar in a 15 mL heavy wall pressure glass vial reactor capped with a screw top and a Viton-O® ring. The reactor vial is placed in an oil bath preheated to 173° C. to achieve temperature of the liquid mixture inside the reactor of 150° C. The vial was submerged in an oil bath to heat the reaction mixture to 150° C., as measured by a thermocouple inserted directly into the reaction mixture through a modified screw cap. The reaction mixture is stirred at 200 rpm for the duration of the reaction time. After the desired reaction time, the vial is removed from the oil bath and quenched in cold water. The reactor vials are centrifuged at 1400 rpm for 15 minutes, and the supernatant liquid is extracted with a syringe for further analysis. The remaining solids are washed with acetone and centrifuged at 1400 rpm for 15 minutes twice. The acetone is removed with a syringe and the solids are dried in an oven at 65° C. overnight and their weight is measured.

Concentrations of water-soluble products are determined by High Performance Liquid Chromatography (HPLC) analysis of the liquid recovered from centrifugation. The glucose yield is calculated based on the following formula:

$$\frac{{m}_{g} * {M}_{g u}}{{m}_{c} * {M}_{g}} * 100 \%$$

where m_{c} is mass of cellulose, m_{g} is mass of glucose determined by HPLC, M_{gu} is molecular weight of glucose unit in cellulose, and M_{g} is molecular weight of glucose.

### Salted-Promoted Hydrolysis

Cellulose was hydrolyzed with different aqueous salt solutions. Specifically, cellulose was first ball-milled for 50 minutes to reduce crystallinity. Then 0.1 g of ball-milled cellulose was mixed with 2 mL aqueous salt solution which consists of 0.05 M HCl and 0.5 M salt. Several salts were tested: NH₄Cl (Sigma Aldrich®), CaCl₂) (Sigma Aldrich®), KCl (Sigma Aldrich®), LiCl (Millipore Sigma®), C(NH₂)₃Cl (Sigma Aldrich®). The reaction was carried out in a 15 mL heavy wall pressure glass tube (ChemGlass®) sealed by a screw cap with a Viton® O-ring seal. The stirring bar was set as 200 rpm and reaction temperature was set as 150° C. heated in an oil bath. The reaction time was set as 0.5 h, 1 h, 2 h, 3 h and 4 h. After the desired reaction time, the pressure tube was removed from oil bath and cooled down in ice bath. After the cooling, resultant liquid suspension was transferred to 50 mL centrifuge glass tube. Further liquid-solid separation was conducted in a centrifuge for 20 minutes at 2500 rpm. After centrifuge, top clear liquid was transferred to glass vials for further HPLC analysis. The residue solid was washed by acetone twice and dried at 65° C. overnight.

Several control tests were conducted to analyze the inorganic salt effects. Cellulose hydrolysis with 0.05 M hydrochloride acid was conducted in similar fashion except removing the salt; the effect of salts on cellulose hydrolysis was carried out by mixing 0.1 g cellulose with 0.5 M guanidinium chloride and reacting for 4 hours under 150° C.; avicel-101 was used as crystalline cellulose, and it was reacted with 0.05 M hydrochloride acid and 0.5M guanidinium chloride (Sigma Aldrich®) for 4 hours at 150° C. All other analysis remained the same as inorganic salt-hydrochloride acid-cellulose reaction.

### Centrifuge Process and Sample Collection

In the examples described below, a Thermo Scientific Sorvall Legend RT+ Centrifuge was used to separate the liquid from the solid. The following settings were used for each of the samples. The ramp up and ramp down speed was set to 5. The rpm was set to 3000 rpm. The time was set to 10 minutes. The temperature was set to 25° C. The samples were loaded into the centrifuge and ran for 10 minutes. The liquid was extracted from the vial and saved for analysis. The solid was washed with 2 mL of acetone and shaken to ensure even washing of the solid. The vial was placed in the centrifuge for 10 more minutes. The liquid was extracted and removed to the proper waste. 2 mL of acetone was added to the sample again and shaken to ensure even washing. The vial was placed in the centrifuge for a final 10 minutes. The liquid was extracted and disposed of in the proper waste. The solid was then placed into a 60° C. oven to dry overnight.

### X-Ray Diffractometer (XRD)

X-Ray Diffractometer, also known as XRD, was an instrument used to measure the crystallinity of the cellulose using a Rigaku® Geigerflex diffractometer. CuKα radiation was emitted at 37.5 kV and 25 mA. A step size of 0.05° was used with 1 second accumulation time.

Diffractograms of the different samples were compared after the area was normalized and baseline subtraction. Crystallinity of the cellulose was calculated by using the peak height method, a method developed by Segal (Segal et al. 1959, An empirical method for estimating the degree of crystallinity of native cellulose using the X-Ray diffractometer. Text Res J 29 (10): 786-794.). This method takes height ratio between the intensity of the crystalline peak and the total intensity after the subtraction of the non-crystalline signal. The following equation was used:

$$CI ( \% ) = \frac{{I}_{200} - {I}_{A}}{{I}_{200}} \times 100 \%$$

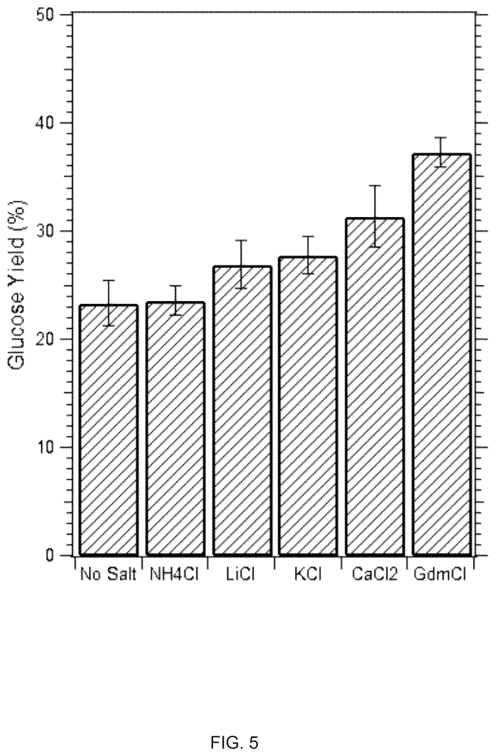

CI (crystallinity index) is the calculated crystallinity in percent (%), 1200 is the maximum intensity of the peak that corresponding to the plane with the Miller indices 200 at the 2Θ angle at 22.5°. IA is the intensity of diffraction of the background scatter (amorphous), at the 20 angle of about 18.3° in the valley between the peaks (Terinte, N., Ibbett, R. and Schuster, K. C., 2011. Overview on native cellulose and microcrystalline cellulose I structure studied by X-ray diffraction (WAXD): Comparison between measurement techniques. Lenzinger Berichte, 89 (1), pp. 118-131.). FIG. 5 shows the XRD results for raw MCC and the 50-minute ball-milled MCC. The highest peak, marked with the 200 Miller indices, is the highly crystalline portion of cellulose I. The peak is drastically reduced after the 50-minute ball milling. From this observation, we can see that the post process of ball milling greatly increases the amorphous regions in cellulose I.

### High Performance Liquid Chromatography (HPLC)

Liquid products were analyzed with High Performance Liquid Chromatography (HPLC, Shimadzu® LC-40 model). A diode array detector (DAD) was used for organic acids and furanic compounds and a refractive index detector (RID) for carbohydrate detection. Bio-Rad Aminex HPX-87H (Phenomenex) was used for product separation. The mobile phase was 5 mM sulfuric acid for preventing bacteria growing. The mobile phase flow rate was 0.6 mL/min and analyzing temperature was 35° C. A series of standard glucose solutions was prepared for obtaining calibration curve, which are 0.25, 0.5, 1, 2 and 5 g/L.

### Raman Microscopy

Raman spectral analysis of cellulose samples was carried out with a Horiba Xplora™ Raman Microscope using 785 nm excitation laser and 10× Olympus magnification lens. The acquisition range was set from 300 cm-1 to 1600 cm⁻¹.

### Example 1: Cation Effect on Suppression of Cellulose Recrystallization

In one example, samples containing cellulose were ball milled for 50 minutes in an aqueous solution at 150° C. for 120 minutes (2 hours). One of the samples was mixed with water alone while the remaining samples were mixed with aqueous salt solutions (LiCl, KCl, CaCl₂), and GdmCl). The crystallinity of the cellulose in each sample was measured. Table 1 below compares the crystallinity (%) of cellulose in each of the samples.

TABLE 1
Crystallinity (%) of Cellulose

| | Segal Crystallinity |
|---|---|
| Sample | Index (%) |
| Water | 88.2% |
| NH₄Cl (aq) | 86.6 ± 1.1% |
| LiCl (aq) | 80.1 ± 2.2% |
| KCl (aq) | 81.1 ± 1.8% |
| CaCl₂ (aq) | 78.9 ± 1.0% |
| GdmCl (aq) | 79.1 ± 2.1% |

As shown, water alone and NH₄Cl in aqueous solution provide the highest crystallinity of cellulose. In contrast, the aqueous salt solutions (LiCl, KCl, CaCl₂), and GdmCl) reduce the crystallinity index of cellulose.

It is noted that the suppression of recrystallization was mainly from the cations in the salt, since the anion in all samples was chloride.

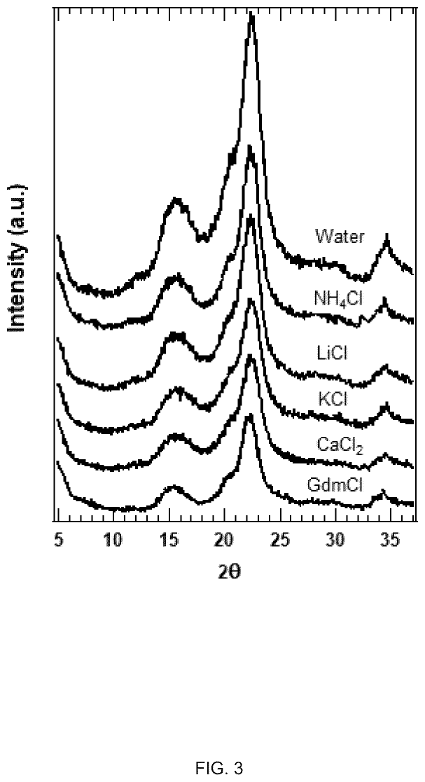

FIG. 3 shows the XRD results for each of the samples. The Segal crystallinity index calculations show that chaotropic salts, such as guanidinium chloride (Sigma Aldrich®), suppressed the cellulose recrystallization the most and kosmotropic salts, such as ammonium chloride, suppressed cellulose recrystallization the least.

### Example 2: Anion Effect on Suppression of Cellulose Recrystallization

In another example, experiments were performed to determine whether the anion influenced the suppression of cellulose recrystallization. This was performed with the 50-minute ball-milled cellulose with 0.5M aqueous salt solution at 150° C. for 1-hour. Table 2 below compares the crystallinity (%) of cellulose in each of the samples.

TABLE 2
Crystallinity (%) of Cellulose

| | Segal Crystallinity |
|---|---|
| Sample | Index (%) |
| Water | 91.86% |
| Na₂CO₃ | 91.85% |
| C₂H₃NaO₂ | 91.64% |
| NaCl | 90.91% |
| NaH₂PO₄ | 88.11% |
| NaOH | 89.02% |

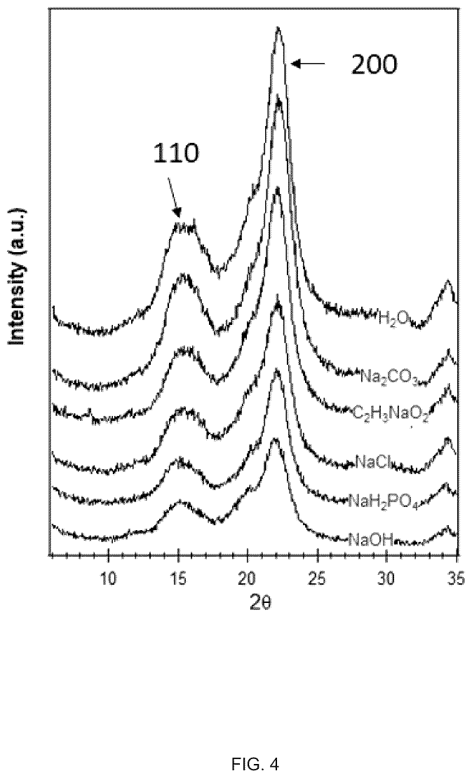

FIG. 4 shows the XRD results for each of the samples. In all the aqueous salt solutions, the 200_cellulose plane and the 110_cellulose plane recrystallized, but at different kinetic speeds, marked as “200” and “110 on the XRD chart. After 1 hour, the sample with no salt (water) caused cellulose to restore its crystallinity completely. Sodium Hydroxide suppressed cellulose recrystallization the most, followed by Sodium Phosphate, Sodium Chloride, Sodium Acetate, and Sodium Carbonate, respectively. Sodium Carbonate and Sodium Acetate fully restored cellulose crystallinity post heat treatment and did not influence suppressing cellulose recrystallization.

### Example 3: Glucose Yields of Fixed-Anion Salt Selections

In another example, the glucose yields from hydrolyzing cellulose in the presence of a chaotropic salt were measured. Samples containing cellulose were ball milled for 50 minutes. Subsequently, the samples were mixed with a 500 mM aqueous salt solution at 150° C. for 240 minutes (4 hours). Hydrochloric acid was added to hydrolyze the cellulose.

The resulting mixtures were analyzed with high performance liquid chromatography (HPLC, Shimadzu LC-40 model) to determine glucose yields. FIG. 5 shows the results of the HPLC analysis. Adding salts increased the glucose yield. Without any salt, Hydrochloric Acid only produced 23% glucose yield (no-salt). Guanidinium chloride produced the highest glucose yield with around 37%. Calcium Chloride produced around 31%, Potassium Chloride produced around 27.5%, Lithium Chloride produced around 26.5%, and Ammonium Chloride produced around 23%. The results of the glucose yields agree with the recrystallization suppressing performance.

HPLC was also performed on the salt selections that were ran for 2 hours with no acid. As expected, no glucose was produced. This indicates that the increase of glucose yield post hydrolysis is due to the salts ability to suppress the cellulose recrystallization.

### Example 4: Glucose Yields of Fixed-Cation Salt Selections

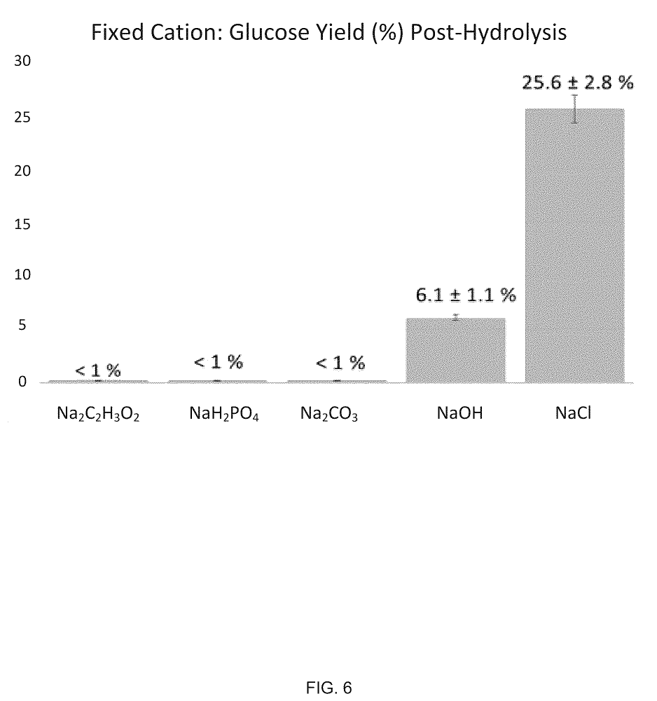

In another example, glucose yields for fixed-cation salts were obtained. FIG. 6 shows the glucose yield results. Sodium Chloride produced the highest glucose yields with around 25%. Sodium Hydroxide produced a glucose yield of about 6%. Sodium carbonate, sodium phosphate, and sodium acetate produced glucose yields of less than 1%. The results of the fixed-cation data showed that the anions from the various salt selections possibly had a reaction with the acid.

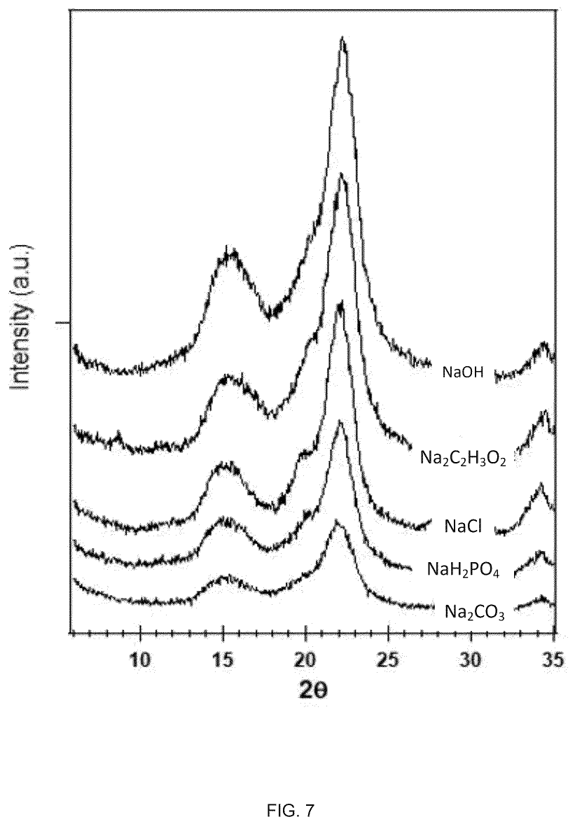

XRD was performed on these samples to see if the recrystallization supported that possibility. Post hydrolysis, sodium hydroxide suppressed cellulose recrystallization the least, while sodium carbonate suppressed cellulose recrystallization the most. The difference in the XRD results, as seen in FIG. 7, show that some of the salt interacted with the acid during hydrolysis, limiting the amount of salt to suppress crystallinity.

As shown in Table 3, the pKa value of the acid produced from the salt-acid interaction for each sample determines how tightly the proton is held by a Brønsted acid. The lower the pKa of a Brønsted acid, the more easily it will give up its proton. Likewise, the higher the pKa of a Brønsted acid, the tighter the proton is held. Sodium chloride produced a glucose yield of 25.6%. Hydrochloric acid has a pKa value of −9.3, making it a strong acid. This acid fully disassociates in water, creating a proton and a chlorine anion. The proton can continue to attack the glycosidic bonds to produce more glucose units. On the other hand, if the Brønsted acid has a larger value, such as those of carbonic acid (6.37) and acetic acid (4.75), then the proton is held on tighter and cannot attack the cellulose to produce glucose, resulting in less than 1% production.

TABLE 3
Glucose Yields by Salt

| | | Glucose Yields |
|---|---|---|
| Table: Acid-Base Reactions | pKa | (%) |
| NaCl + HCl → NaCl + HCl | −9.3 | 25.6% |
| Strong Acid: | | |
| HCl + H₂O → H₃O++ Cl— | | |
| NaOH + HCl → NaCl + H₂O | 14.0 | 6.1% |
| Na₂CO₃ + 2HCl → 2NaCl + H₂CO₃ | 6.37:10.329 | <1% |
| H₂CO₃ + H₂O→HCO₃— + H₃O+ | | |
| HCO₃— + H₂O → CO₃— + H₃O+ | | |
| CH3COONa + HCl → NaCl + | 4.75 | <1% |
| CH3COOH | | |
| Weak acid: | | |
| CH3COOH + H₂O ↔ CH3COO— + H+ | | |
| NaH₂PO₄ + 2HCl → NaHCl₂ + H₃PO₄ | 2.12 | <1% |

The salts' ability to suppress crystallinity correlates with its performance in glucose yield, showing that the performance of salts agrees with the Hofmeister series. The XRD data supported the hypothesis that salting-in salts, such as Guanidinium Chloride, would suppress cellulose recrystallization, while salting-out salts, such as Ammonium Chloride, would support cellulose recrystallization. In the examples described above, Guanidinium Chloride produced the highest glucose yields after hydrolysis, while ammonium chloride produced the least amount of glucose for the fixed-anion salt selections.

Although Sodium hydroxide suppressed cellulose crystallinity the most, it produced only 6% glucose due to the salt-acid interaction during hydrolysis. Sodium Chloride performed particularly well out of the fixed-cation salts due to its minimal salt-acid interaction.

### Example 5: Molecular Dynamic Simulations

A molecular dynamic simulation was performed using GROMACS software, version 5.0 (http://www.gromacs.org). The initial cellulose chain structure is a 8-glucose-chain 1-β cellulose fibril and 4 cellulose fibril chains was randomly solvated in the simulation box pre-defined as 6 nm cubic box. Packmol was used to assemble the solute molecules that consists of the 1M guanidine hydrochloride or ammonium chloride and 4 cellulose fibril chains. The cutoff radius of nonbonded interactions was set to 1.2 A and the particle mesh Ewald (PME) summation method was used to calculate the electrostatic potential with periodic boundary conditions. Charmm36 force field is used to extract parameters.

The simulation was performed in the following steps:
1) Packmol package was used to prepare solvent box that consists of 1 M salt (guanidine hydrochloride or ammonium chloride).
2) The solvent box was solvated by TIP3P water molecules. Then four cellulose fibril chains were inserted into the box randomly.
3) The system was equilibrated to minimize the energy.
4) The system was equilibrated under NVT ensemble for 200 ps.
   Formula 1 is applied to calculate . . .

$$V = {\sum }_{bonds} {{k}_{r} ( r - {r}_{eq} )}^{2} + {\sum }_{angles} {{k}_{θ} ( θ - {θ}_{eq} )}^{2} + {\sum }_{\text{?}} \frac{{V}_{\text{?}}}{2} [ 1 - cos ( \text{?} ϕ - \text{?} ) ] + {\sum }_{\text{?}} {{K}^{\text{?}} ( x - {x}_{eq} )}^{2} + {\sum }_{\text{?}} \frac{\text{?}}{\text{?}} + {\sum }_{bonds} \text{?} [ {( \frac{\text{?}}{\text{?}} )}^{\text{?}} - {( \frac{\text{?}}{\text{?}} )}^{\text{?}} ] \qquad Formula 1$$

$$\text{?} \text{indicates text missing or illegible when filed}$$

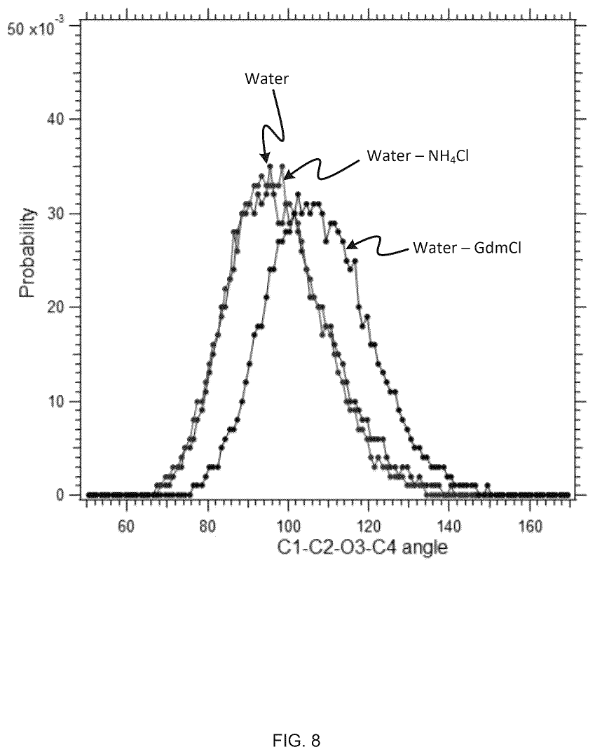

As shown in FIG. 8, three systems were simulated with the GROMACS software: 1M guanidinium chloride with 4 cellulose chains, 1M ammonium chloride with 4 cellulose chains and pure cellulose chains interacting with water. Dihedral angles for labeled C1-C2-O3-C4 was computed for all three samples.

Interestingly, angle distribution for cellulose-water system is centered around 94°, which is almost identical to water-ammonium chloride-cellulose mixture whose angle is around 96°, suggesting that ammonium chloride effect on cellulose might be like water's effect on cellulose. However, when the water-guanidinium chloride-cellulose mixture was simulated, the angles become slightly spreaded suggesting that the bonds are influenced and relaxed to some extent once mixing with guanidinium chloride.

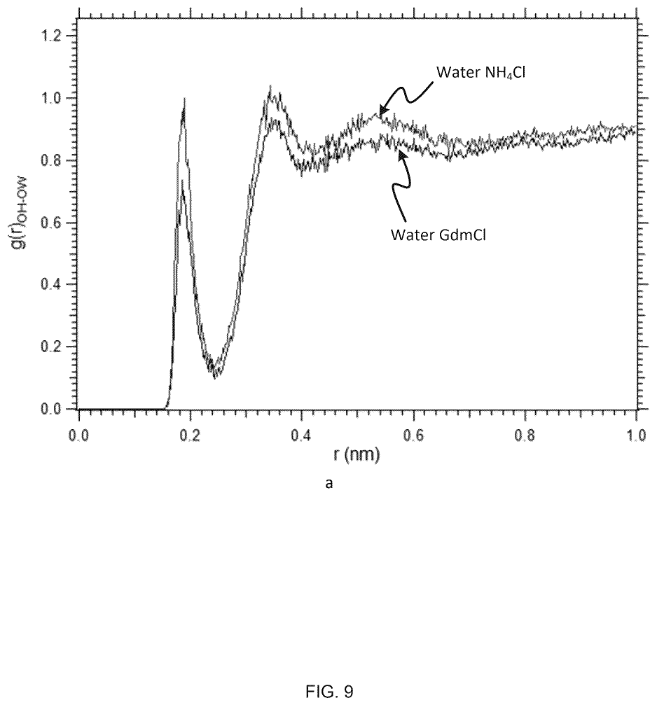

The GROMACS software was further used to determine the radial distribution of —OH in cellulose with water's oxygen for both ammonium chloride and guanidinium chloride. As shown in FIG. 9, the first hydration peak is at around 0.2 nm, which is hydrogen bonding. As separation increases, number density around-OH varies for these two salts. Number density of guanidinium chloride is slightly greater than ammonium chloride, suggesting that water molecules are pushed away by guanidinium cations. But the differentiation is small, indicating that both ammonium cations and guanidinium cations may have the accessibility to interact with interior part of cellulose, which may interfere its crystalline structures.

The results shown in FIG. 9 demonstrate that the interaction between-OH group of cellulose with water is an important type of interaction contributing to cellulose crystallization. Hydrogen bonding between-OH and water helps cellulose maintain the amorphous phase as it prevents cellulose from self-binding by hydrogen bonds, a phenomenon called the hydrophobic effect.

By maintaining cellulose in an amorphous state, the chaotropic salt enhances access to the glucose subunits by solvents, catalysts, and enzymes. This in turn increases the efficiency of cellulose hydrolysis. Cellulose hydrolyzed in the presence of a chaotropic salt yields more glucose in less time. Furthermore, since the chaotropic salt allows hydrolysis to proceed at lower temperatures and less harsh conditions.

### Example 6: Guanidinium Chloride-Mediated Enzymatic Hydrolysis of Microcrystalline Cellulose

### 1. Materials

### 1.1 Equipment

Weighing scale.
Vibratory ball mill
Orbital shaker.
Incubator.
Centrifuge.
Hot plate.
HPLC machine equipped with a refractive index detector (RID) and an HPLC column (Phenomenex Rezex ROA-Organic Acid H+ (8%))

### 1.2 Reagents and Supplies

Microcrystalline cellulose.
Guanidinium chloride (or guanidine hydrochloride).
Sodium chloride.
Citric acid monohydrate.
Sodium hydroxide.
Sodium azide.
Cellic CTec2 (cellulase enzyme blend).
Sulfuric acid.
25 ml Erlenmeyer flasks.
Adjustable micropipettes.
10 ml Glass test tubes.
15 ml Centrifuge tubes.
3 ml Disposable syringes, fitted with 0.22 μm syringe filters
Autosampler vials

### 2. Procedure

Weigh 1 g microcrystalline cellulose (MCC), place it in the cylinder of the vibratory mill containing three stainless steel balls, and ball mill for 50 minutes to obtain ball-milled cellulose (MCC50).

Prepare stock solutions of guanidinium chloride (GdmCl, 5 M concentration), sodium chloride (NaCl, 5 M concentration), and sodium azide (3% w/v) in deionized water.

Prepare a 50 mM solution of citric acid in deionized water and add drops of 1 M sodium hydroxide solution to titrate the pH to 5. This will result in a 50 mM citrate buffer (pH 5).

Prepare the reaction mixture by adding the following reagents into 25 ml Erlenmeyer flasks.

For a 5 ml reaction volume (for control hydrolysis experiments without GdmCl), add the following into the 25 ml flasks (in the order below):
0.5 g of biomass (either MCC or MCC50)-10% w/v solids loading
4.4595 ml of the citrate buffer.
0.5 ml of sodium azide-0.3% w/v
40.5 μl of the cellulase enzyme blend (Cellic CTec2)—enzyme dose of 20 FPU/g biomass

For a 5 ml reaction volume with the addition of different concentrations of GdmCl (or NaCl controls), this will affect only the volume of citrate buffer to be added. All the other reagents (biomass, sodium azide, and enzyme) will have the same amounts found in [0070]. Modify the volumes of citrate buffer and add GC (or NaCl) as shown in Table 4:

TABLE 4

| Conc. | Volume | Volume |
|---|---|---|
| of GdmCl | of citrate | of GdmCl |
| or NaCl (M) | buffer (ml) | or NaCl (μl) |
| 0.05 | 4.4095 | 50 |
| 0.1 | 4.3595 | 100 |
| 0.2 | 4.2595 | 200 |
| 0.4 | 4.0595 | 400 |
| 0.8 | 3.6595 | 800 |
| 1.0 | 3.4595 | 1000 |

Cover the reactor flasks and place them in an incubator shaker set at 150 rpm shaking speed and 50° C. temperature for enzymatic hydrolysis for 24 hours.

Remove the flasks after 24 hours of hydrolysis and transfer the contents into 10 ml glass test tubes.

Place the test tubes in vigorously boiling water for 8 minutes to inactivate the enzymes.

Remove the test tubes from the boiling water and allow them to cool to room temperature.

Transfer the contents of the test tubes into 15 ml centrifuge tubes and centrifuge at 4000 rpm for 15 minutes.

Collect the supernatants (hydrolysates) and filter them through 0.22 μm syringe filters into autosampler vials for HPLC analysis.

Collect the solid residues and dry them for further analysis.

Calibrate the RID detector response by analyzing glucose samples with known concentrations (0.25, 0.5, 1, 1.5, 2, 5, 7, and 10 g/L) to generate a glucose calibration curve.

Analyze the hydrolysates for their glucose content using the HPLC machine under the following conditions:
Injection volume: 20 μl.
Mobile phase: 5 mM H2SO4.
Flow rate of mobile phase: 0.6 ml/min.
Column temperature: 35° C.
Run time: 20 minutes

Integrate the peaks of the chromatogram to obtain the area under the peak corresponding to the glucose retention time.

Insert the areas into the calibration curve to interpolate the glucose concentrations of the hydrolysates.

### 3. Summary of GC-Glucose Yield %

The results of the experiments described in Example 6 are shown below in Tables 5, 6, 7, and 8.

TABLE 5

| | | 0.05M | 0.1M | 0.2M | 0.4M | 0.8M | 1.0M |
|---|---|---|---|---|---|---|---|
| Time | | GdmCl - | GdmCl - | GdmCl - | GdmCl - | GdmCl - | GdmCl - |
| (hr) | MCC | MCC | MCC | MCC | MCC | MCC | MCC |
| 3 | 2.77 ± | 18.51 ± | 19.44 ± | 19.10 ± | 5.13 ± | 5.57 ± | 2.84 ± |
| | 0.48 | 2.56 | 0.97 | 1.39 | 0.31 | 0.25 | 0.27 |
| 6 | 7.28 ± | 24.02 ± | 20.91 ± | 25.01 ± | 9.06 ± | 7.51 ± | 3.50 ± |
| | 2.68 | 2.59 | 1.05 | 3.14 | 0.39 | 0.39 | 0.07 |
| 12 | 10.55 ± | 34.73 ± | 35.26 ± | 32.67 ± | 11.16 ± | 6.40 ± | 3.32 ± |
| | 0.83 | 0.34 | 7.61 | 1.45 | 0.81 | 1.14 | 0.12 |
| 24 | 10.02 ± | 40.73 ± | 39.29 ± | 38.50 ± | 13.05 ± | 7.86 ± | 3.53 ± |
| | 3.14 | 0.59 | 0.57 | 4.62 | 0.93 | 0.05 | 0.54 |

TABLE 6

| | | 0.05M | 0.1M | 0.2M | 0.4M | 0.8M | 1.0M |
|---|---|---|---|---|---|---|---|
| Time | | GdmCl - | GdmCl - | GdmCl - | GdmCl - | GdmCl - | GdmCl - |
| (hr) | MCC50 | MCC50 | MCC50 | MCC50 | MCC50 | MCC50 | MCC50 |
| 3 | 6.29 ± | 20.35 ± | 24.38 ± | 33.83 ± | 11.86 ± | 11.68 ± | 7.68 ± |
| | 1.38 | 0.44 | 1.35 | 1.44 | 0.92 | 0.53 | 0.83 |
| 6 | 9.86 ± | 35.05 ± | 39.91 ± | 50.38 ± | 20.78 ± | 19.64 ± | 10.49 ± |
| | 0.32 | 3.90 | 1.34 | 0.46 | 0.95 | 0.70 | 0.76 |
| 12 | 17.68 ± | 54.52 ± | 73.08 ± | 72.14 ± | 29.87 ± | 23.27 ± | 13.72 ± |
| | 0.99 | 0.78 | 3.51 | 0.84 | 0.24 | 1.66 | 0.98 |
| 24 | 21.11 ± | 62.70 ± | 93.43 ± | 97.15 ± | 41.17 ± | 22.60 ± | 14.62 ± |
| | 2.36 | 0.96 | 3.00 | 5.55 | 2.66 | 4.82 | 0.60 |

TABLE 7

| Time | | 0.2M NaCl - | 0.4M NaCl - | 1.0M NaCl - |
|---|---|---|---|---|
| (hr) | MCC | MCC | MCC | MCC |
| 3 | 2.77 ± 0.48 | 7.25 ± 0.26 | 8.09 ± 1.97 | 8.28 ± 0.43 |
| 6 | 7.28 ± 2.68 | 9.68 ± 1.23 | 11.87 ± 0.62 | 9.99 ± 0.85 |
| 12 | 10.55 ± 0.83 | 15.11 ± 0.34 | 16.27 ± 0.17 | 12.24 ± 0.71 |
| 24 | 10.02 ± 3.14 | 20.97 ± 2.25 | 19.87 ± 0.27 | 12.87 ± 4.93 |

TABLE 8

| Time | | 0.2M NaCl - | 0.4M NaCl - | 1.0M NaCl - |
|---|---|---|---|---|
| (hr) | MCC50 | MCC50 | MCC50 | MCC50 |
| 3 | 6.29 ± 1.38 | 10.61 ± 0.97 | 11.46 ± 0.67 | 8.97 ± 1.40 |
| 6 | 9.86 ± 0.32 | 17.78 ± 1.33 | 20.18 ± 1.02 | 14.52 ± 0.53 |
| 12 | 17.68 ± 0.99 | 26.63 ± 1.28 | 28.54 ± 4.14 | 14.80 ± 3.38 |
| 24 | 21.11 ± 2.36 | 33.98 ± 0.94 | 36.15 ± 1.27 | 16.15 ± 0.47 |

The results show that mechanochemical pretreatment is an effective, waste-free method to increase the sugar yields obtainable by enzyme hydrolysis of biomass compared with untreated biomass. The mechanism involves cellulose amorphization and increasing the enzyme-accessible surface area of biomass. The energy required for mechanochemical pretreatment was estimated using several different approaches to be on the order of 0.5 to 5.6 MJ kg-1, comparable to that required for dilute acid pretreatment and much less than the combustion energy of the ethanol product. Mechanochemical pretreatment, enzyme hydrolysis, and fermentation could be used to decarbonize transportation. Depending on the severity of the mechanochemical pretreatment, biomass cultivation could satisfy a significant portion of the world's gasoline needs.

The scope of the claims should not be limited by the embodiments set forth in the above examples but should be given the broadest interpretation consistent with the description as a whole.

1. A method of hydrolyzing cellulose, the method comprising:
providing cellulose to be hydrolyzed;
applying a stabilizing salt to the cellulose to form a reaction mixture, the stabilizing salt configured to suppress recrystallization of amorphous cellulose in the reaction mixture; and
hydrolyzing the amorphous cellulose to obtain fermentable sugars, wherein hydrolysis occurs in the presence of the stabilizing salt to suppress recrystallization of the amorphous cellulose in the reaction mixture during the hydrolysis.

2. The method of claim 1, wherein the stabilizing salt is selected according to its Hofmeister series order.

3. The method of claim 1, wherein the stabilizing salt comprises a low-order Hofmeister series salt.

4. The method of claim 1, wherein the stabilizing salt comprises a chaotropic salt.

5. The method of claim 1, wherein the stabilizing salt is selected from a list consisting of: guanidine salts, aminoguanidine, salts of alkali metals, and alkaline earth metal salts.

6. The method of claim 1, wherein the stabilizing salt comprises guanidinium chloride.

7. The method of claim 1, wherein the stabilizing salt forms a part of an aqueous solution to be applied to the cellulose.

8. The method of claim 7, wherein a concentration of the stabilizing salt is between 0.05M to 1 M.

9. The method of claim 7, wherein a concentration of the stabilizing salt is between 0.1 M and 1 M.

10. The method of claim 7, wherein a concentration of the stabilizing salt is between 0.05 M and 0.4 M.

11. The method of claim 7, further comprising selecting a concentration of the stabilizing salt according to a selected method of hydrolyzing the amorphous cellulose.

12. The method of claim 1, further comprising selecting the stabilizing salt according to a selected method of hydrolyzing the amorphous cellulose.

13. The method of claim 1, further comprising decrystallizing the cellulose to form the amorphous cellulose.

14. The method of claim 13, wherein decrystallizing the cellulose comprises ball milling.

15. The method of claim 1, wherein hydrolyzing the cellulose comprises enzymatic hydrolysis.

16. The method of claim 1, wherein hydrolyzing the cellulose comprises acid hydrolysis.

17. The method of claim 1, further comprising:
isolating the fermentable sugars from the reaction mixture; and
fermenting a remainder of the reaction mixture to recover the stabilizing salt.
